(19) United States
(12) Patent Application Publication
(10) Pub. No.: US 2023/0044904 A1
(43) Pub. Date: Feb. 9, 2023

(54) MAGNETIC SAFETY GLASSES

(71) Applicant: Daniel Walker, Placerville, CA (US)

(72) Inventor: Daniel Walker, Placerville, CA (US)

(21) Appl. No.: 17/961,425

(22) Filed: Oct. 6, 2022

Related U.S. Application Data
(63) Continuation-in-part of application No. 16/778,332, filed on Jan. 31, 2020.
(60) Provisional application No. 62/801,042, filed on Feb. 4, 2019.

(51) Int. Cl. A61F 9/02; F16M 13/02

(52) U.S. Cl. CPC A61F 9/029; F16M 13/022

(57) ABSTRACT

Safety glasses have two magnets located near temple arm hinges so that surfaces of both magnets become coplanar when the temple arm pieces close. The magnets provide two separated areas of coupling that allow the safety glasses to adhere to a surface and stay in place when jarring forces are present. The safety glasses can be stored on magnetically active surfaces or other surfaces with adaptive tabs and may be kept close to where they will be used and in a visible location as a safety reminder or safe location to protect the safety glasses.

### CROSS-REFERENCE TO RELATED APPLICATIONS

This patent document a continuation-in-part and claims benefit of the earlier filing date of U.S. patent Ser. No. 16/778,332, filed Jan. 31, 2020, which claims benefit of the earlier filing date of U.S. Provisional Pat. App. No. 62/801,042, filed Feb. 4, 2019, both of which are hereby incorporated by reference in their entirety.

### BACKGROUND

Some current solutions for storage of eyewear attempt to externally protect or guard the eyeglasses with a case or pouch, but these solutions fail to meet the needs of safety glasses used in an industrial setting because the cases or pouches are often forgotten or are not used. Lanyards can hold eyeglasses around one's neck, but such positioning of safety glasses may be dangerous because the safety glasses or lanyards could be entangled in motorized equipment and because lanyards may place the safety glasses in the way of other protective equipment. U.S. Pat. No. 6,568,805 discloses a solution for carrying eyeglasses that uses magnets in an attractive magnetic communication and impart a gripping force on an article of clothing, but these solutions are similarly unable to meet the needs of safety glasses used in industry because the coupling mechanism generally isn't configured to and isn't strong enough to keep the glasses stable during storage in a vehicle or on machinery.

### SUMMARY

It is desirable to have safety glasses that may be stored at the location where the safety glasses will be used while keeping the safety glasses out of harm's way. Furthermore, it is desirable to provide reminders that encourage proper use of safety glasses before operating or using many tools, machinery, chemicals, or other items that may be found in an industrial location. Still further, it is desirable to have safety glasses that are in good condition and that do not obstruct one's sight. The devices and methods disclosed herein may advantageously fill these needs by providing an integrated storage that allows a user to store the safety glasses where the safety glasses may next be used and where the safety glasses are in view of users. The stored safety glasses may thus serve as a reminder where the safety glasses are needed. The safety glasses systems and storage methods disclosed herein also allow for systematic storage that reduces the chance of misplacing safety glasses.

Disclosed are safety glasses or other protective eyewear, which may include the following components a lens or lenses, a frame to support the lens or lenses, two temple arm pieces, two hinges, attaching the temple arm pieces to the frame or the lenses, and two magnets mounted in or on the temple arm pieces. (These components may be connected to one another with clips, tabs, screws, or adhesives or are manufactured in a way as to join pieces as one.) The hinges allow the temple arm pieces to be opened for use and closed for storage, and the magnets may be disposed on the temple arm pieces adjacent to the hinges and may be flush with or recessed within the temple arm pieces so that magnetic surfaces on the temple arms become substantially coplanar when the temple arm pieces are closed toward the frame.

The safety glasses may provide one or more of the following: health benefits, increased compliance to workforce safety protocols, display benefits in a store or workplace, reduction of replacement cost.

The safety glasses are unique when compared with other known devices and solutions because the safety glasses provide two points of coupling for storage and may be ANSI certified for use as proper protective equipment. The safety glasses are also structurally unique due to the presence of two magnets that may couple two separated areas in a single plane, which allows for storage of the safety glasses on equipment that require use of the safety glasses for safe operation, where the safety glasses are visible, and/or where the safety glasses are out of harm's way.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The magnetic safety glasses may, however, be embodied in many different forms and should not be construed as being limited to the embodiments set forth herein. Rather, these embodiments are provided by way of illustration only and so that this disclosure will be thorough, complete and fully convey understanding to those skilled in the art.

### BRIEF DESCRIPTION OF THE DRAWINGS

FIG. 1 shows a perspective view of safety glasses with temple magnets in accordance with one example of the present disclosure.

FIG. 2 shows a top view of safety glasses when open with temple magnets in accordance with one example of the present disclosure.

FIG. 3 shows a top view of safety glasses when closed with temple arm pieces closed and positioning the magnets to affix the safety glasses to iron or another ferromagnetic surface.

FIG. 4 shows a top view of safety glasses when closed with temple magnets and intervening tabs or adapters that permit the safety glasses to adhere to any surface.

The drawings illustrate examples for the purpose of explanation and are not of the invention itself. Use of the same reference symbols in different figures indicates similar or identical items.

### DETAILED DESCRIPTION

The present invention is directed to safety glasses and methods for securing or storing safety that are not being worn and particularly to safety glasses and methods for using safety glasses including magnets capable of holding the safety glasses on a magnetically active surface or with adapters to most any surface.

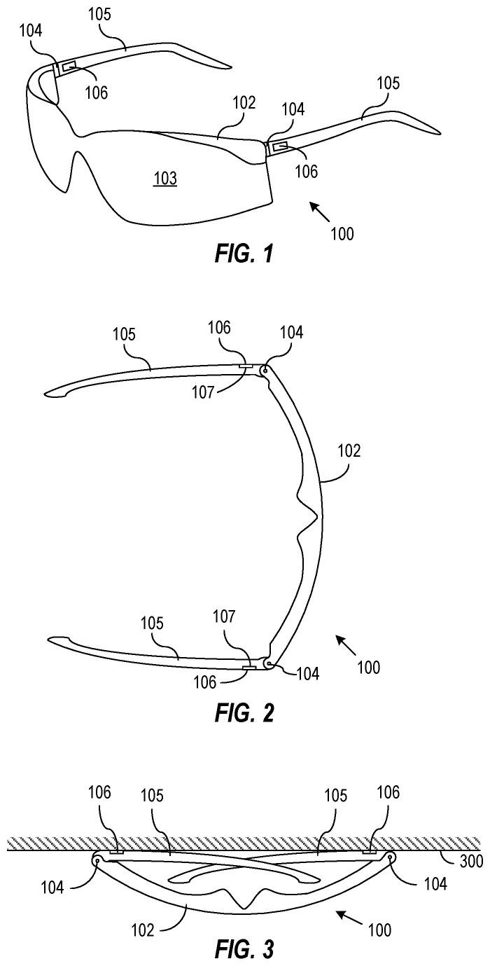

FIGS. 1 and 2 respectively show perspective and top views of safety glasses 100 in accordance with an example implementation of the present disclosure. In the illustrated implementation, safety glasses 100 include a frame 102, a lens or pair of lenses 103, a pair of hinges 104; a pair of temple arm pieces 105, and a pair of magnets 106 that are mounted on temple arm pieces 105. The term lens here is not limited to optical elements that provide effects such as focusing or filtering of light but is used herein in a general sense to include any protective devices through which a user may see. In particular, lens or lenses 103 may be made of a durable clear plastic or any other conventional construction for safety glasses 100 without any focusing or filtering power or may provide focusing or filtering, for example, for magnification, vision correction, or protection from bright light. Safety glasses 100 illustrate an example in which a frame-lens assembly includes frame 102 and lens(es) 103, but in alternative implementations of safety glasses, a frame-lens assembly may be an integrated or one-piece structure that hinges 104 attach to respective temple arm pieces 105.

Two integral magnets 106 are mounted on, embedded in, or otherwise joined to temple arm pieces 105 at the temple areas that are near to the hinges 104. For example, magnets 106 may be embedded in temple arm pieces 105 at locations that are closer to the proximal or hinged ends of the respective temple arm pieces 105 than to the distal or free ends of temple arm pieces 105. Further, magnets 106 may be mounted at locations within 1 inch or less from respective hinges 104. Magnets 106 may provide magnetic surfaces that are flush with or recessed relative to the surface of temple arm pieces 105, so that magnets 106 do not require a bulge in temple arm pieces 106. Accordingly, temple arm pieces 105 with magnets 106 may be more compact and do not have a bulge that might catch on other objects when safety glasses 100 are handled or moved.

Magnets 106 may, for example, be rare earth magnets such as neodymium magnets and samarium—cobalt magnets, which are strong, lightweight, permanent magnets, and magnets 106 may be set in cavities 107 formed in temple arm pieces 105 so that surfaces of magnets 106 are flush with or recessed in smooth surfaces of temple arm pieces 105. Magnets 106 may be oriented to present the same magnetic pole, e.g., both north or both south, or opposite poles, e.g., one north and the other south, outward from temple arm pieces 105. The positioning of magnets 105 on temple arm pieces prevents magnets 105 from coming into contact with each other throughout the entire range of rotation of temple arm pieces 105 about hinges 104.

FIGS. 1 and 2 particularly show safety glasses 100 when temple arm pieces 105 are unfolded or in the open position for use. In the open position of FIGS. 1 and 2, a user can wear safety glasses 100 in a conventional manner, for example, with a bridge or nose pads of frame 102 resting on a user's nose and portions of temple arm pieces 105 resting on the user's ears.

All components of safety glasses 100 combine to form a coupling system that allows a consumer or user to store or secure safety glasses 100 where he or she needs and out of harm's way. For example, magnets 106 may be adjacent to respective temple arm hinges 104 and located so that magnetic surfaces lie in a flat plane with each other when temple arm pieces are closed toward frame 102. Magnets 106 further provide two separated points of contact for securing safety glasses 100 on a flat surface when the temple arm pieces 105 are in the closed position. FIG. 3 shows a top view of the safety glasses 100 of FIGS. 1 and 2 when temple arm pieces 105 are folded toward frame 102 (into the closed position) for storage of safety glasses 100. The positioning of magnets 106 allows for coupling of safety glasses 100 to magnetically active materials (i.e., to magnets and unmagnetized ferromagnetic materials such as iron, steel, and many alloys) in a storage surface 300. Storage surface 300 may be a flat or planar surface or may have other shapes, for example, a cylindrical surface or any surface that provides two areas to which magnets 106 can attach. Magnetically active surfaces such as storage surface 300 may be found in many places in industrial or workshop environments. For example, storage surface 300 may be a surface of a toolbox, a power tool, a sawhorse, a firearm, a refrigerator, a locker, a work bench, many types of machinery, or other items. Magnets 106 are positioned so they do not contact each other or significantly interact with each other magnetically in the closed position of FIG. 3 or in any other position of temple arm pieces 105 including the open position of FIGS. 1 and 2.

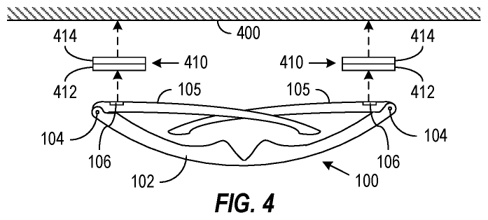

Safety glasses 100 may also be attached to surfaces that are not magnetically active. FIG. 4 illustrates a storage surface 400 that is not magnetically active by itself but employs one or more tabs 410 that have a magnetically active layer 412 capable of coupling to magnets 106 and a fastener layer 414 capable of coupling storage surface 400. Storage surface 400 may, for example, be a non-metal surface such as a wood or plastic surface, a non-magnetic metal surface such as an aluminum surface, or may be a surface covered with fabric, upholstery, or other material. Storage surface 400 may, for example, be a surface of a visor in an automobile, an automobile dashboard, an aluminum housing, a plastic housing, a hat, a suitcase, or a plastic toolbox. Magnetically active layer 412 may, for example, include an iron or other magnetically active material that is not magnetized or may employ a magnet with the proper orientation to attract one or both of magnets 106. Fastener layer 414 may employ one or more of many different methods of coupling to various surfaces 400. For example, adhesive layer 414 may employ double-sided tape, a layer of adhesive on the back of magnetically active layer 412, or at least a portion of a Velcro® fastener or other hook-and-loop fastener or touch fastener, e.g., the hook side of a hook-and-loop fastener capable of adhering to some fabrics. FIG. 4 illustrates a system or attachment method employing two tabs 410. Alternative configurations may employ any number of tabs 410 that collectively provide two magnetically active areas for magnets 106 to attach. For example, a single tab may have sufficient length to provide two magnetically active attachment areas at a separation that matches the separation of magnets 106 in the closed configuration of FIG. 4.

An advantage of safety glasses 100 is that both temple arm pieces 105 contain magnets 106 providing two separated attachment areas and thereby allowing for secure storage when surface 300 or 400 is vertical or even while safety glasses 100 are upside down under a horizontal surface 300 or 400. Magnets 106 can couple to separated areas in a single plane, which keeps the safety glasses 100 from swinging or pivoting around a central or other single point, for example, in response to jarring forces or gravity creating a torque on an unbalanced mounting. Integral magnets 106 also allow safety glasses 100 to be stored in almost any location and particularly anywhere one deems beneficial. This capability can keep safety glasses 100 out of harm's way, conveniently located for use, or in a conspicuous location where safety glasses 100 serve as a reminder to use safety equipment. For example, safety glasses 100 may be mounted on a bench or floor standing power tool such as a grinder, drill press, or a table saw where a user will see safety glasses 100 and be reminded to use safety glasses 100 as soon as the user approaches the tool.

Although particular implementations have been disclosed, these implementations are only examples and should not be taken as limitations. Various adaptations and combinations of features of the implementations disclosed are within the scope of the following claims.

What is claimed is:

1. A safety glasses system comprising:
a frame-lenses assembly;
two temple arm pieces;
two hinges respectively attaching hinged ends of the temple arm pieces to the frame-lenses assembly; and
two magnets respectively mounted in or on the two temple arm pieces and at locations closer to respective the hinges than to free ends of the temple arm pieces, the two magnets being located to remain separated from each other while the temple arm pieces are opened and closed.

2. The system of claim 1, wherein the two magnets are positioned such that surfaces of the two magnets respectively lie in two separated areas of a plane when the temple arm pieces are folded toward the frame.

3. The system of claim 1, further comprising one or more tabs, each of the one or more tabs having a magnetically active layer and a fastener layer, the fastener layer being configured to adhere to a surface that is not magnetically active.

4. The system of claim 3, wherein the fastener layer comprises double-sided tape, a layer of adhesive, or at least a portion of a hook-and-loop fastener on a back of the magnetically active layer.

5. The system of claim 3, wherein the one or more tabs comprises two of the tabs.

6. The system of claim 1, wherein the magnets are embedded in the respective temple arm pieces with surfaces of the magnets being flush with surfaces of the temple arm pieces.

7. The system of claim 6, wherein the temple arm pieces include cavities in which contain respective magnets.

8. The system of claim 1, wherein the magnets are embedded in the respective temple arm pieces with the magnets being recessed below surfaces of the temple arm pieces.

9. A method for securing safety glasses, the method comprising:
closing first and second temple arm pieces of the safety glasses, the first and second temple arm pieces respectively including first and second magnets closer to hinged ends of the first and second temple arm pieces, wherein hinges connect the hinged ends of the first and second temple arm pieces to a frame-lens assembly of the safety glasses; and
with the first and second arm pieces closed, placing the safety glasses so that a first surface of the first magnet and a second surface of the second magnet respectively adhere to two separated magnetically active areas on a storage surface.

10. The method of claim 9, further comprising an operator wearing the safety glasses while operating machinery before placing the safety glasses on the storage surface, wherein the storage surface is a surface of the machinery.

11. The method of claim 9, wherein the storage surface is a surface of machinery, and wherein the method further comprises an operator removing the safety glasses from the storage surface and wearing the safety glasses while operating the machinery.

12. The method of claim 9, wherein placing of the safety glasses positions the safety glasses on machinery in a location where the safety glasses are in a line of sight from a position of a user approaching the machinery for use.

13. The method of claim 9, wherein the storage surface is a non-magnetic surface, and the method further comprises adhering one or more tabs to the storage surface, the one or more tabs including the magnetically active areas.

14. The method of claim 9, wherein the storage surface comprises a surface of a power tool, the method further comprising an operator removing the safety glasses from the surface of the power tool and using the safety glasses for a safe operation of the power tool.

15. The method of claim 9, wherein the storage surface is on one of a toolbox, a power tool, a sawhorse, a firearm, a refrigerator, a locker, and a work bench.

16. The method of claim 9, further comprising attaching adhering first and second tabs to form the two separated magnetically active areas on the storage surface, each of the first and second tab including a magnetically active layer and a fastener layer, the fastener layer being configured to adhere to a surface that is not magnetically active.
